(19) United States
(12) Patent Application Publication
(10) Pub. No.: US 2023/0019357 A1
(43) Pub. Date: Jan. 19, 2023

(54) ENDOSCOPE

(71) Applicant: OLYMPUS CORPORATION, Tokyo (JP)

(72) Inventors: Keita MITSUHASHI, Tokyo (JP); Tsukasa OTA, Tokyo (JP); Takuto YOSHINAGA, Tokyo (JP); Wataru MATSUURA, Tokyo (JP); Motohiko SUZUKI, Sagamihara-shi (JP)

(73) Assignee: OLYMPUS CORPORATION, Tokyo (JP)

(21) Appl. No.: 17/954,683

(22) Filed: Sep. 28, 2022

Related U.S. Application Data
(63) Continuation of application No. PCT/JP2020/014685, filed on Mar. 30, 2020.

(51) Int. Cl. A61B 1/005; A61B 1/00

(52) U.S. Cl. CPC A61B 1/0057; A61B 1/00042

(57) ABSTRACT

An endoscope includes a pulley provided in an operation portion, having an outer diameter larger than an inner diameter of an endoscope insertion portion, and configured to wind a wire connected to a bending portion; and a wire guard that is arranged to cover a predetermined range of an outer peripheral portion of the pulley, and includes a guide wall arranged to extend within a range of a radius of the pulley from a contact point position at which the wire in contact with the pulley is separated from the pulley in a longitudinal axis direction of the operation portion. The wire guard includes two members each formed in a substantially semicircular plate shape, and coupling parts formed in arc shapes extending from both end portions of the two members and configured to couple the two members.

### CROSS REFERENCE TO RELATED APPLICATION

This application is a continuation application of PCT/JP2020/014685 filed on Mar. 30, 2020, the entire contents of which are incorporated herein by this reference.

### BACKGROUND OF THE INVENTION

### 1. Field of the Invention

The present invention relates to an endoscope operation portion including a bending portion provided in an insertion portion, and an endoscope.

### 2. Description of the Related Art

As is well known, endoscopes are widely used for observation, treatment, and the like of the interior of the body (body cavity) of a living body, or for inspection, repair, and the like of the interior of industrial plant facilities. The above-mentioned endoscope has an elongated insertion portion to be inserted into a body cavity, an abdominal cavity, a duct, or the like of the subject. It is well known that the insertion portion of the endoscope is provided with a bending portion at a leading end portion thereof in order to enhance capability of insertion into the subject to be examined.

The bending portion provided in the insertion portion is driven by a bending operation wire that is pulled or slackened in accordance with an operation amount of a bending operation lever provided in an operation portion, and a bending amount is variably operated in a predetermined direction.

For example, Japanese Unexamined Patent Application Publication No. 2009-165722 discloses a configuration of an endoscope in which a belt engaged with a sprocket inside an operation portion is coupled to a bending operation wire, and the sprocket is rotated by a bending operation to pull and drive the bending operation wire.

In the above-mentioned endoscope of related art, a roller tensioner for eliminating slack of the belt and the bending operation wire provided inside the operation portion is disposed in contact with a smooth surface of the belt, thereby making it possible to eliminate disconnection of a linear member wound on a winding wheel, achieve a simple and lightweight structure compared to an endoscope using a chain, and obtain excellent responsiveness of the bending operation.

Further, for example, International Publication No. WO 2018/034021 discloses a configuration of an endoscope in which a bending wire extended from a thin insertion portion is connected to a pulley having a larger diameter than that of the insertion portion, so as to perform a bending operation.

In the above-mentioned endoscope of related art, the bending portion is switched between a long bending state and a short bending state to improve capability of insertion into a deep portion of a complicated body cavity, and to enhance operability in order to perform observation and treatment while maintaining the short bending state.

### SUMMARY OF THE INVENTION

An endoscope according to an aspect of the present invention includes an endoscope insertion portion provided with a bending portion; an operation portion connected with the endoscope insertion portion; an operation member provided in the operation portion and configured to perform bending operation on the bending portion; a pulley provided in the operation portion, having an outer diameter larger than an inner diameter of the endoscope insertion portion, and configured to wind a wire connected to the bending portion; and a wire guard that is arranged to cover a predetermined range of an outer peripheral portion of the pulley, and includes a guide wall arranged to extend within a range of a radius of the pulley from a contact point position at which the wire in contact with the pulley is separated from the pulley in a longitudinal axis direction of the operation portion, wherein the wire guard includes two members each formed in a substantially semicircular plate shape, and coupling parts formed in arc shapes extending from both end portions of the two members and configured to couple the two members.

### BRIEF DESCRIPTION OF THE DRAWINGS

FIG. 1 is a plan view illustrating a configuration of an endoscope apparatus according to the present embodiment.

FIG. 2 is a plan view illustrating a configuration of a baby endoscope according to the present embodiment.

FIG. 3 is a side view illustrating a configuration of a baby endoscope according to the present embodiment.

FIG. 4 is a partial cross-sectional view illustrating an internal configuration of an operation portion according to the present embodiment.

FIG. 5 is a plan view illustrating an internal configuration of an operation portion according to the present embodiment.

FIG. 6 is an exploded perspective view illustrating a configuration of a pulley unit according to the present embodiment.

FIG. 7 is a cross-sectional view illustrating a configuration of a pulley unit according to the present embodiment.

FIG. 8 is a partially enlarged cross-sectional view illustrating a configuration of a pulley unit according to the present embodiment.

FIG. 9 is an exploded perspective view illustrating a configuration in which a pulley unit and the like are attached to an outer casing member of an operation portion according to the present embodiment.

FIG. 10 is a partial cross-sectional view explaining a configuration of a wire guard of a pulley unit according to the present embodiment.

FIG. 11 is a diagram explaining action of a wire guard in a state where a pulley is rotated according to the present embodiment.

FIG. 12 is a diagram explaining action of a wire guard in a state where a pulley is rotated to the maximum according to the present embodiment.

FIG. 13 is a perspective view of a clamp of a wire clamp seen from one direction according to the present embodiment.

FIG. 14 is a perspective view of the clamp of the wire clamp seen from another direction according to the present embodiment.

FIG. 15 is a perspective view illustrating a configuration of a clamp base of a wire clamp according to the present embodiment.

FIG. 16 is an exploded perspective view illustrating a configuration in which a wire clamp is attached to a pulley according to the present embodiment.

FIG. 17 is an exploded perspective view explaining a state in which a bending operation wire is fixed to a wire clamp according to the present embodiment.

FIG. 18 is a cross-sectional view of a state in which a bending operation wire is fixed to a wire clamp according to the present embodiment.

FIG. 19 is a perspective view illustrating an outer casing member of an operation portion according to a first reference example.

FIG. 20 is a diagram obtained by enlarging a portion indicated by a circle XX in FIG. 19 for explaining a configuration in which watertightness of a mouthpiece of a channel is maintained by a sealing member according to the first reference example.

FIG. 21 is a perspective view illustrating a configuration of a distal end portion main body according to a second reference example.

FIG. 22 is a front view illustrating a configuration of a distal end portion main body according to the second reference example.

FIG. 23 is a cross-sectional view illustrating a configuration of a distal end portion main body where an image pickup module is attached in an opening hole according to the second reference example.

FIG. 24 is a partial plan view illustrating a state where an image pickup module is attached in an opening hole of a distal end portion main body according to the second reference example.

FIG. 25 is a cross-sectional view illustrating a configuration of a leading end portion of an insertion portion according to a third reference example.

FIG. 26 is a perspective view illustrating a configuration of a leading end portion of an insertion portion according to a fourth reference example.

### DETAILED DESCRIPTION OF THE PREFERRED EMBODIMENT(S)

Hereinafter, an endoscope according to an aspect of the present invention will be described with reference to the accompanying drawings. In the following description, the drawings based on the respective embodiments are schematic; it should be noted that the relationship between the thickness and the width of each portion, the ratio of the thicknesses of the respective portions, and the like are different from the actual ones, and there are cases where the portions in which the relationship and the ratio of dimensions are mutually different are included across the drawings.

The endoscope in the following configuration description exemplifies a so-called soft endoscope having a flexible insertion portion to be inserted into a body cavity such as the stomach, small intestine, bile duct, pancreatic duct, or large intestine from the bronchus, urinary organ, or esophagus of a living body, but the endoscope in the following configuration description is not limited thereto, and may be applied to a so-called rigid endoscope with a rigid insertion portion including a bending portion to be used in surgery. Note that the endoscope herein exemplifies a mother-and-baby type endoscope including a mother endoscope and a baby endoscope, which are medical devices.

An endoscope apparatus 1 serving as a medical device according to the present embodiment includes a mother endoscope 10 and a baby endoscope 20 to be fixed to the mother endoscope 10. In this case, the baby endoscope 20 is, for example, a single-use (disposable) medical device to be discarded after use.

The mother endoscope 10 includes an insertion portion 11 formed in an elongated shape to be inserted into the subject, and an operation portion 12 provided with a bend preventing portion for coupling a proximal end side of the insertion portion 11.

The insertion portion 11 is formed having an outside diameter of about 10 mm and a length of about 1.2 m, and is configured by coupling a distal end portion main body 13, a bending portion 14, and a flexible tube portion 15 in sequence from the distal end side.

A treatment instrument insertion channel, which is a duct (not illustrated), is formed inside the insertion portion 11. The treatment instrument insertion channel extends passing through from a treatment instrument insertion portion 16 provided in the operation portion 12 to the distal end portion main body 13. A disposable forceps plug 16a is attached to the treatment instrument insertion portion 16 in a detachable manner.

In addition to the treatment instrument and the like, for example, an insertion portion 21 of the baby endoscope 20 may be inserted into the treatment instrument insertion channel.

Inside the distal end portion main body 13, there is provided a treatment instrument raising base (forceps elevator) (not illustrated) for raising a treatment instrument, the insertion portion 21 of the baby endoscope 20, or the like that is inserted into the treatment instrument insertion channel (not illustrated) and protrudes from an opening provided at a side of the leading end portion thereof. That is, the mother endoscope 10 is a side-view/slant-view type endoscope.

Although not illustrated, an image pickup unit incorporating, for example, an image sensor of an objective optical system, CCD, CMOS or the like, an illumination optical system for radiating illumination light transmitted by a light-guide bundle, and the like are disposed in the distal end portion main body 13 of the insertion portion 11 or the operation portion 12.

The operation portion 12 includes various operation members necessary for operating the mother endoscope 10, a universal cable 17 for connecting with an endoscope unit (not illustrated) configured to control the mother endoscope 10, and the like. An endoscope connector 17a attachable to and detachable from an external device (not illustrated) such as a light source apparatus is provided at an extended end of the universal cable 17.

The flexible tube portion 15 of the insertion portion 11 is configured by a flexible tubular member that can be bent passively. An image pickup cable, a light-guide bundle, the treatment instalment insertion channel, and an air/water feeding tube (none of them are illustrated) are inserted into the flexible tube portion 15. As the operation members of the operation portion 12, a pair of bending operation knobs 18 for performing a bending operation on the bending portion 14, an operation lever 19 for inverting and raising the treatment instrument raising base, and the like are provided.

The bending portion 14 of the insertion portion 11 is allowed to actively bend in all circumferential directions about a longitudinal axis (insertion axis) of the insertion portion 11 including up-down/left-right directions in accordance with rotation operation input of the pair of bending operation knobs 18 by a user who is an operator, or the like.

The configuration of the baby endoscope 20 as a medical device used together with the mother endoscope 10 according to the present embodiment will be described in detail below.

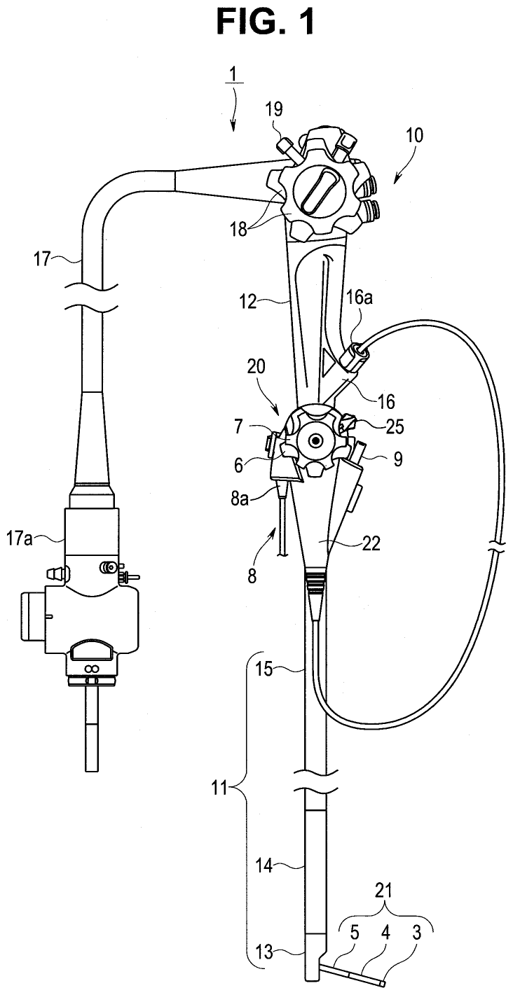

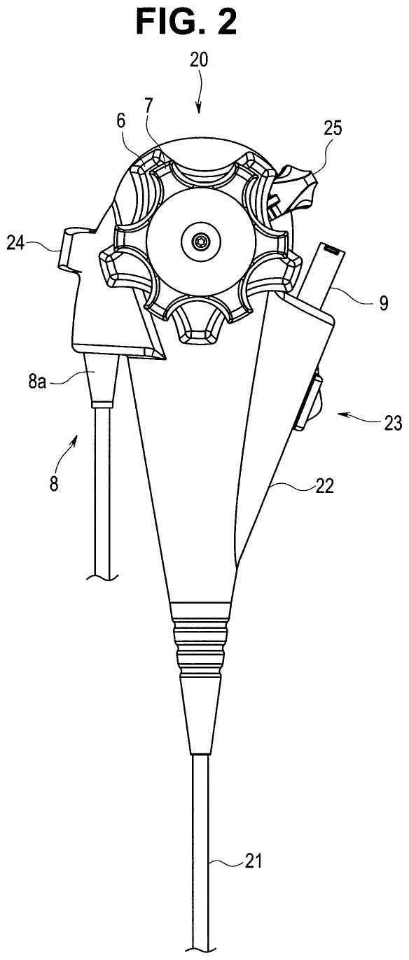

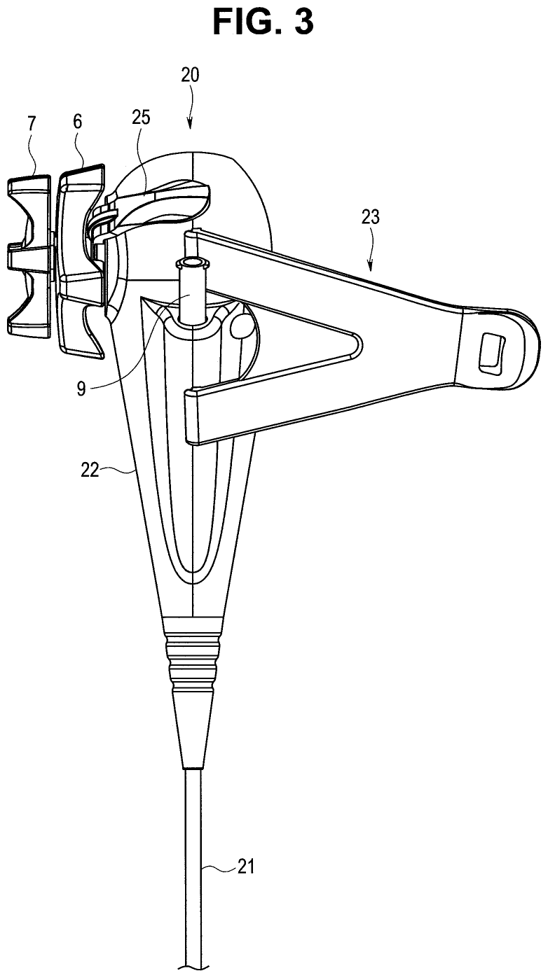

The baby endoscope 20 illustrated in FIGS. 1 to 3 includes the insertion portion 21 formed in an elongated shape and an operation portion 22 coupled to the proximal end side of the insertion portion 21.

The insertion portion 21 is formed having an outside diameter of about 3 to 4 mm and a length of about 2 m, and is configured by coupling a distal end portion main body 3, in which an observation window, an illumination window, and the like (not illustrated) are disposed, a bending portion 4, and a flexible tube portion 5 in sequence from the distal end side.

An image pickup unit incorporating an image sensor of an objective optical system, CCD, CMOS or the like (not illustrated), an LED light source for radiating illumination light, an illumination optical system, and the like are disposed in the distal end portion main body 3 of the insertion portion 21 or the operation portion 22, similar to the mother endoscope 10.

The operation portion 22 includes a pair of bending operation knobs 6 and 7 for performing the bending operation on the bending portion 4, and a brake lever 25 for fixing rotational positions of the pair of bending operation knobs 6 and 7, and further includes various operation members (not illustrated) necessary for operating the baby endoscope 20.

In addition, the operation portion 22 of the baby endoscope 20 is provided with a hook portion 24 as a locking portion configured to lock a fixing band 23 in a detachable manner. The fixing band 23 is a flexible belt-like member, and is formed of resin such as silicone rubber having a substantially triangular outer shape, for example, a letter A-like shape.

The baby endoscope 20 is attached to the mother endoscope 10 in such a manner that the fixing band 23 is wound around the operation portion 12 of the mother endoscope 10 and is hooked on the hook portion 24, whereby an outer casing portion of the operation portion 22 is kept to be in contact with the operation portion 12 of the mother endoscope 10.

As in the mother endoscope 10, the bending portion 4 of the insertion portion 21 is allowed to actively bend in all circumferential directions about a longitudinal axis (insertion axis) of the insertion portion 21 including the up-down/left-right directions in accordance with rotation operation input of the pair of bending operation knobs 6 and 7 by the user as an operator, or the like.

The insertion portion 21 of the baby endoscope 20 is provided with a channel (not illustrated) for injecting a contrast medium agent or the like. A distal end of the channel is opened in the distal end portion main body 3, and the proximal end of the channel communicates with a mouthpiece 9 provided in the operation portion 22.

A cable 8 for the image pickup, power supply and the like, and tubes such as an air/water feeding tube and a suction tube (not illustrated) are provided extending from the operation portion 22.

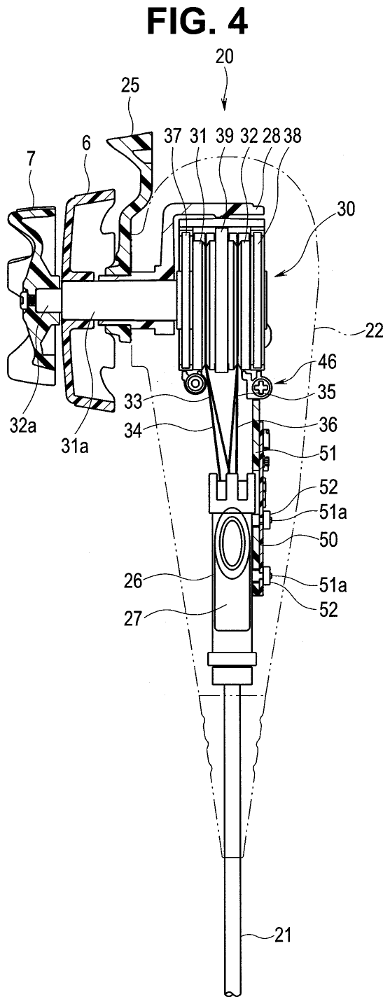

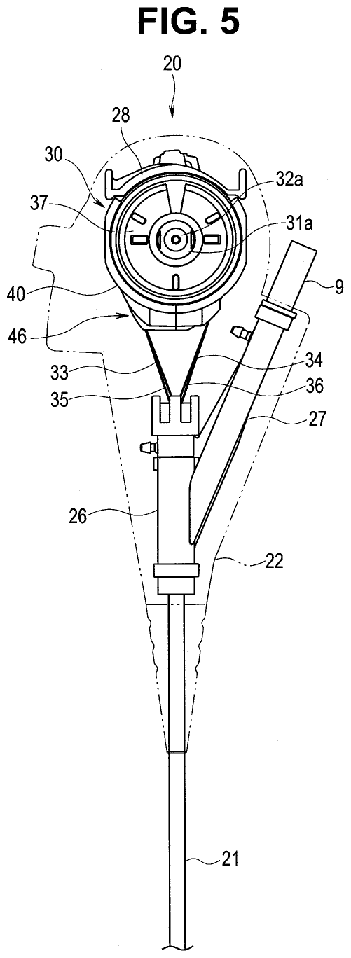

As illustrated in FIGS. 4 and 5, a pulley unit 30 connected to the bending operation knobs 6 and 7 operated when bending the bending portion 4 of the insertion portion 21 is provided in the operation portion 22 of the baby endoscope 20. The pulley unit 30 includes two pulleys 31 and 32, which will be described later, each having an outer diameter larger than an inner diameter of the tubular-shaped insertion portion 21.

In one pulley 31, a hub 31a, which is a rotation shall center, is formed in a tubular body, and a hub 32a, which is a rotation shaft center of the other pulley 32, is inserted into the hub 31a to protrude from an end portion of the hub 31a.

The hub 31a of the one pulley 31 is connected with the bending operation knob 6 on the large diameter side close to the operation portion 22, and is configured to perform the bending operation on the bending portion 4 of the insertion portion 21 in the up-down direction (see FIG. 4). That is, bending operation wires 33 and 34 for bending the bending portion 4 in the up-down direction are wound on the pulley 31.

The hub 32a of the other pulley 32 is connected with the bending operation knob 7 on the small diameter side separated from the operation portion 22 and stacked on the bending operation knob 6, and is configured to perform the bending operation on the bending portion 4 in the left-right direction (see FIG. 4). That is, bending operation wires 35 and 36 for bending the bending portion 4 in the left-right direction are wound on the pulley 32.

The outer peripheral portions of the two pulleys 31 and 32 are each pressed by a brake member 28 connected to the brake lever 25 by a tilting operation of the brake lever 25, and consequently the rotational positions thereof are fixed. Thus, the bending state of the bending portion 4 is maintained. The brake member 28 is disposed to pass through the root portion of the hub 31a of the pulley 31 and rotate.

The four bending operation wires 33 to 36 extending from the two pulleys 31 and 32 toward the insertion portion 21 are inserted into a tubular member 26 connected with the proximal end of the insertion portion 21. The four bending operation wires 33 to 36 are each inserted into a coil tube (not illustrated) and disposed inside the insertion portion 21, and distal ends thereof are each connected to a bending piece, a bending tube formed of a superelastic alloy material such as nickel titanium (Ni—Ti), and a bending tube such as a multi-lumen tube, which are provided in the bending portion 4 (none of them are illustrated).

The tubular member 26 is held inside the operation portion 22, and is connected with a channel insertion tube 27 provided with the mouthpiece 9 of the channel.

Hereinafter, the configuration of the pulley unit 30 according to the present embodiment will be described in detail with regard to the configuration incorporated in the operation portion 22 of the baby endoscope 20.

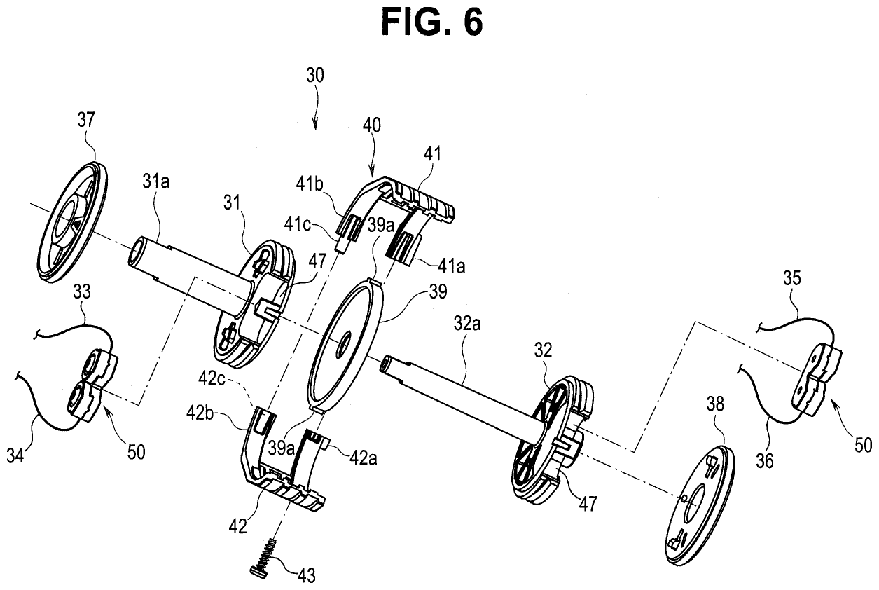

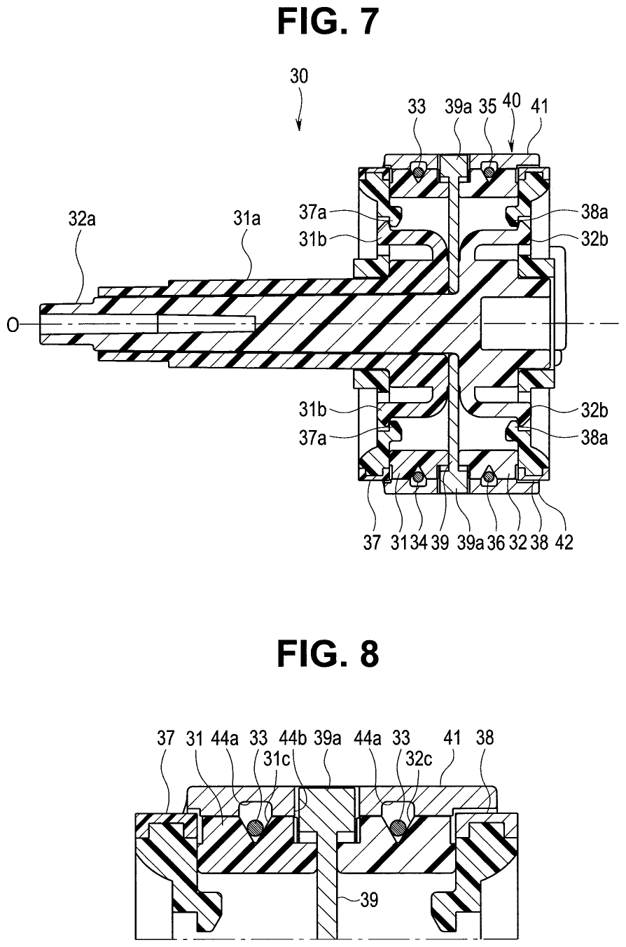

As illustrated in FIGS. 6 and 7, the pulley unit 30 includes the two pulleys 31 and 32 disposed in a rotatable manner, and the bending operation wires 33 to 36 are wound (wound and held) on the two pulleys 31 and 32. End portions of the pair of bending operation wires 33, 34 on the one pulley 31 side and those of the pair of bending operation wires 35, 36 on the other pulley 32 side are respectively fixed to wire clamps 50 configured to engage with recess-shaped clamp arrangement grooves 47 formed in the pulleys 31 and 32, respectively.

The two pulleys 31 and 32 are disposed to sandwich a disk-shaped slide plate 39 having a hole formed in the center thereof, and disk-shaped pulley covers 37, 38 are respectively disposed at the outer sides of the two pulleys 31 and 32.

The two pulley covers 37 and 38 each have a hole formed in the center thereof, and are disposed being stacked in such a manner as to overlap flat surface sides of the two pulleys 31 and 32 sandwiching the slide plate 39, the flat surface sides facing away from each other. That is, the two pulley covers 37 and 38 are provided to sandwich a unit in which the pulley 31, the slide plate 39, and the pulley 32 are stacked in that order.

As illustrated in FIG. 7, the pulleys 31 and 32 are respectively provided with two locking claws 31b and two locking claws 32b at positions symmetrical with respect to a center line (at positions rotating 180° about a central axis O). These locking claws 31b and 32b are formed to protrude from opposing surfaces of the pulleys 31 and 32, to which the pulley covers 37 and 38 are respectively attached.

The pulley covers 37 and 38 each have an opening into which each of the locking claws 31b and 32b of the pulleys 31 and 32 to be attached is fitted, and locking portions 37a and 38a, with which the locking claws 31b and 32b are respectively engaged, are formed in the openings.

In this way, the pulleys 31, 32 and the pulley covers 37, 38 are mechanically bonded by a so-called snap-fit structure that does not require a bonding member such as a screw by engaging the locking claws 31b, 32b with the locking portions 37a, 38a. The wire clamps 50 attached to the pulleys 31 and 32 are pressed and held by the pulley covers 37 and 38 respectively to prevent the detachment thereof.

The pulley unit 30 includes a wire guard 40, which is a guide member disposed to mainly cover a predetermined range of outer peripheral portions of the two overlapping pulleys 31 and 32. As illustrated in FIG. 6, the wire guard 40 includes two guard members 41 and 42 each having a substantially semicircular plate shape.

The two guard members 41 and 42 include belt-like coupling parts 41a, 41b, 42a, and 42b extended in arc shapes from both sides of one end portions of each guard member to constitute a coupling section 46.

The coupling part 41a of the guard member 41 and the coupling part 42a of the guard member 42 are fastened by a screw 43. A pin 41c to be inserted into a pin hole 42c formed in the coupling part 42b of the guard member 42 is provided on the coupling part 41b of the guard member 41, and two coupling parts 41b and 42b are pin-coupled.

As described above, the wire guard 40 is disposed to cover the outer peripheral portions of the two pulleys 31 and 32, where the two guard members 41 and 42 are screw-fastened and pin-coupled. The wire guard 40 is attached such that the coupling section 46 including the coupling parts 41a, 41b, 42a, and 42b is located on the distal end side, which is the insertion portion 21 side (see FIGS. 4 and 5).

In this manner, the wire guard 40 may couple the two guard members 41 and 42 without an increase in size in the thickness direction by extending the coupling parts 41a, 41b, 42a, and 42b configuring the coupling section 46 in arc-belt shapes from both the sides of the one end portions. This makes it possible to prevent the pulley unit 30 from being increased in the thickness direction.

In addition, in each of the two guard members 41 and 42, there is formed an engagement hole 44b, with which two protrusions 39a protruding from the outer peripheral portion of the slide plate 39 are engaged in a state where the wire guard 40 is disposed to cover the outer peripheral portions of the two pulleys 31 and 32. (see FIG. 8).

The two protrusions 39a of the slide plate 39 are provided at positions symmetrical with respect to the center line (at positions rotating 180° about the central axis O). The engagement hole 44b is provided in each of the two guard members 41 and 42 while being adjusted to the positions discussed above.

Thus, the two protrusions 39a protruding in the opposite directions respectively engage with the engagement holes 44b in the wire guard 40, so that the slide plate 39 is held by the wire guard 40 so as not to rotate. In the wire guard 40, guide grooves 44a configured to guide the bending operation wires 33 to 36 are formed at positions facing pulley grooves 31c and 32c of the pulleys 31 and 32 on the inner surfaces of the two guard members 41 and 42.

The wire guard 40 is provided such that the inner surfaces of the two guard members 41 and 42 have slight gaps with the outer peripheral surfaces of the pulleys 31 and 32, and the gaps are set to be equal to or smaller than the outer diameters of the bending operation wires 33 to 36. Thus, by providing the wire guard 40, the bending operation wires 33 to 36 are prevented from coming off so as not to be separated from the pulley grooves 31c and 32c of the pulleys 31 and 32.

The wire guard 40 may be provided such that the inner surfaces of the two guard members 41 and 42 are in contact with or pressed against the outer peripheral surfaces of the pulleys 31 and 32 as long as the amount of operation force of the bending operation knobs 6 and 7 is not large (not heavy) when rotating the pulleys 31 and 32.

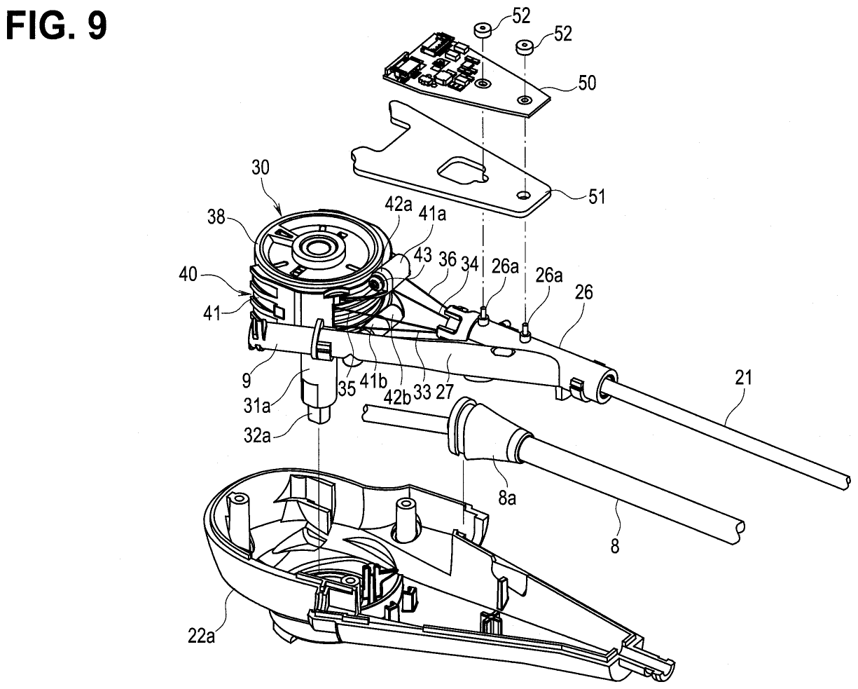

As illustrated in FIG. 9, the pulley unit 30 configured as described above is attached, together with the insertion portion 21 and the tubular member 26, to an outer casing member 22a configuring a half of the operation portion 22 in such a manner that the wire guard 40 does not rotate due to being fitted. The cable 8 is provided with a bend preventing member 8a made of rubber, and the bend preventing member 8a is attached to be engaged with a recessed portion of the outer casing member 22a.

Two spacer protrusions 26a are erected on the outer peripheral portion of the tubular member 26 in a direction being distanced from the pulley unit 30. A protective plate 51 of a magnetic shield and a substrate 50, on which various electronic components are mounted, are disposed to overlap each other on the spacer protrusions 26a, and a nut 52 is attached to each spacer protrusion 26a, so that the protective plate 51 and the substrate 50 are fixed having a predetermined height with respect to the spacer protrusions 26a.

The protective plate 51 is a member configured to block magnetism against the various electronic components on the substrate 50 and prevent the four bending operation wires 33 to 36 from interfering with the substrate 50. That is, the four bending operation wires 33 to 36 are guarded by the protective plate 51 so as not to catch on the substrate 50 in a slackened state.

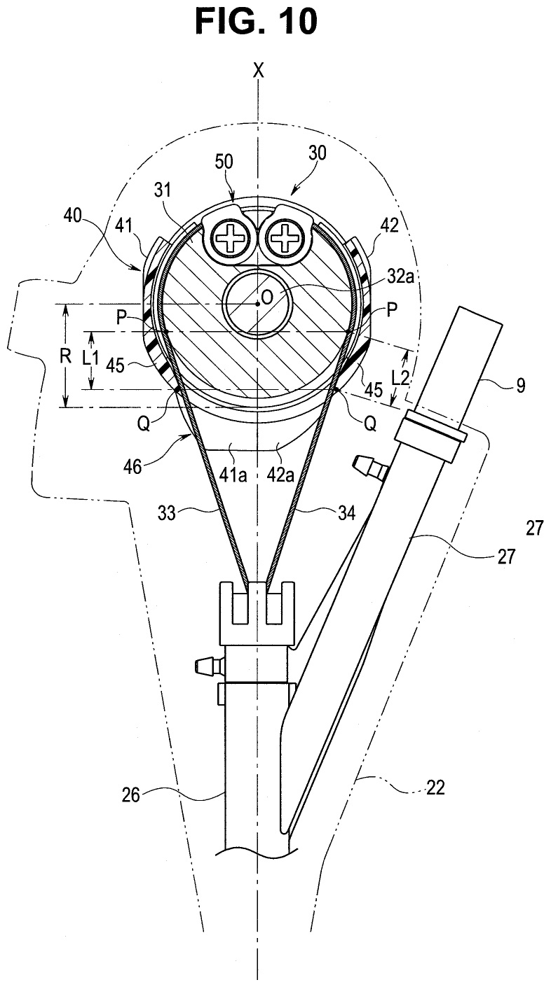

As illustrated in FIG. 10, in the wire guard 40, a wire guide wall 45 extending substantially parallel to a longitudinal axis (an axis in an extending direction) of each of the bending operation wires 33 to 36 (the bending operation wires 35, 36 are not illustrated) of a linear portion directed toward the tubular member 26 in a state in which the bending portion 4 of the insertion portion 21 is not bent (a non-bent state formed in a linear shape) is provided at each leading end portion of the two guard members 41 and 42.

That is, in a state where the wire guard 40 is viewed from the front, the wire guide walls 45 are respectively provided at both left and right side portions. The wire guide walls 45 are each extended from both sides of each of the pulleys 31 and 32 (the pulley 32 is not illustrated) to be disposed being inclined with a predetermined angle on the distal end side directed toward the tubular member 26, as in the insertion form of each of the bending operation wires 33 to 36.

Each of the wire guide walls 45 extends within a radius R of each of the pulleys 31 and 32 on the distal end side directed toward the tubular member 26 along a longitudinal axis X of the operation portion 22 from a central axis O, which is a rotation axis of each of the pulleys 31 and 32.

In other words, the wire guide wall 45 is set in such a manner that a length L1 from a contact point P as a boundary position at which each of the bending operation wires 33 to 36 in contact with the pulleys 31 and 32 is separated from each of the pulleys, to an extended end Q located in a direction orthogonal to a direction along the longitudinal axis X of the operation portion 22 falls within the radius R of each of the pulley 31 and 32, in a state of a portion of each of the bending operation wires 33 to 36 directed toward the tubular member 26 being linear. Since the wire guide wall 45 is inclined at a predetermined angle with respect to the longitudinal axis X, a predetermined length L2 from the contact point P to the extended end Q is set.

Accordingly, each wire guide wall 45 is provided in a range on the proximal end side from the tip to the central axis O of each of the pulleys 31 and 32. The guide groove 44a is also continuously formed on the inner surfaces of the two wire guide walls 45 (see FIG. 8).

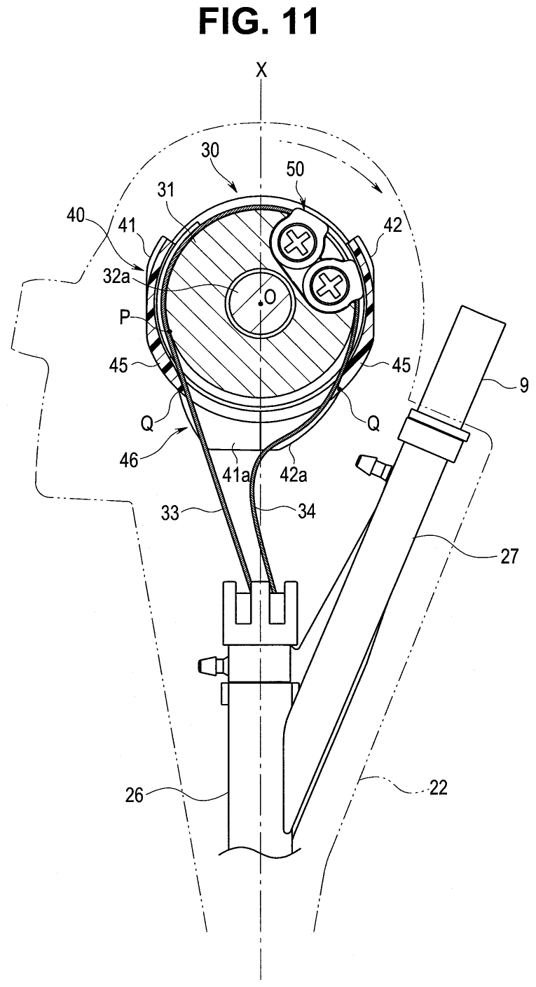

In the baby endoscope 20 described above, when the bending operation knob 6 is rotationally operated in the right direction (clockwise), for example, in order to bend the bending portion 4 of the insertion portion 21 in a predetermined up-down bending direction, the pulley 31 is also rotated clockwise accompanying the knob rotation as illustrated in FIG. 11. Then, one bending operation wire 33 wound on the pulley 31 is pulled, and the other bending operation wire 34 is slackened.

At this time, tension is applied to the one bending operation wire 33 due to being wound on the pulley 31 and being pulled, so that a portion thereof from the contact point P with the pulley 31 to the tubular member 26 comes to be linear. As for the other bending operation wire 34, the winding amount on the pulley 31 is reduced, so that the tension is released and the bending operation wire 34 is in a slackened state. As for the bending operation wire 34 in the slackened state, a portion thereof closest to the pulley 31 and unwound makes contact with the wire guide wall 45 and is guided to bend toward the bending operation wire 33 side on the pulled side.

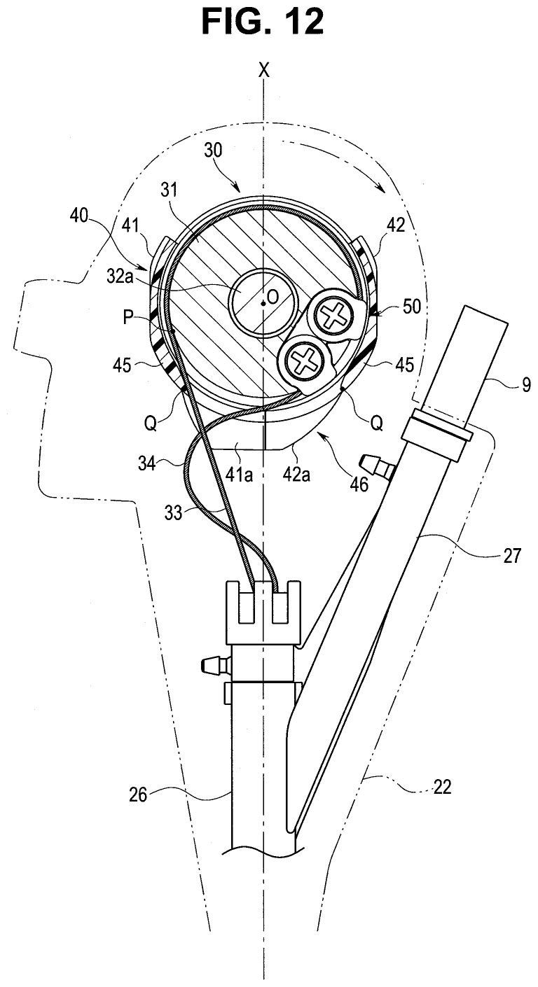

When the bending operation knob 6 is further rotated clockwise, the pulley 31 rotates clockwise, and the slack of the bending operation wire 34 on the slackened side increases. During the process in which the bending portion 4 is bent to the maximum in a predetermined up-down direction as illustrated in FIG. 12, because the wire guide wall 45 for guiding the bending operation wire 33 on the pulled side is not extended in the distal end direction on the tubular member 26 side, the slackened bending operation wire 34 is not wound onto the pulley 31 together with the bending operation wire 33 on the pulled side.

That is, in a case where the wire guide wall 45 is extended longer toward the distal end side beyond the outer peripheral portion of the pulley 31, the slackened bending operation wire 34 is guided along the wire guide wall 45 in a direction in which the bending operation wire 33 on the pulled side directed toward the pulley 31 is wound. Because of this, the slackened bending operation wire 34 may be wound onto the pulley 31.

In this manner, when the slackened bending operation wire 34 is wound onto the pulley 31 from the opposite side (in this case, the left side), there is a risk that a kink is generated due to bending, twisting, or the like and the bending operation wire 34 is broken.

In order to prevent the above incident, an extension range of the wire guide wall 45 formed in the wire guard 40 of the pulley unit 30 is set such that the slackened bending operation wire 34 on the slackened side is not guided toward the pulley 31 by the wire guide wall 45 on the opposite side.

In a case where the bending operation knob 6 is, for example, rotationally operated in the left direction (counterclockwise) and the pulley 31 rotates counterclockwise, and further in a case where the pulley 32 rotates when the bending portion 4 is bent in the left-right direction by the other bending operation knob 7, the same operation is performed and there is exhibited an effect that it is possible to prevent an incident in which each of the bending operation wires 33, 35, and 36 is broken due to the generation of a kink caused by bending, twisting, or the like.

According to the above description, the baby endoscope 20 of the present embodiment has a configuration in which the bending operation wires 33 to 36 for operating the bending portion 4 of the insertion portion 21 may be prevented, with a simple structure, from being wound onto the rotating pulleys 31, 32 and broken, and the like.

The configuration of the pulley unit 30 including the wire guard 40 according to the present embodiment is not limited to the baby endoscope 20, and may be applied to the mother endoscope 10 or various endoscopes including the bending portion in the insertion portion.

### Wire Clamp

The configuration of the wire clamp 50 configured to fix the end portions of the bending operation wires 33 to 36 when the bending operation wires 33 to 36 to be wound on the pulleys 31 and 32 of the pulley unit 30 are attached, will be described in detail below.

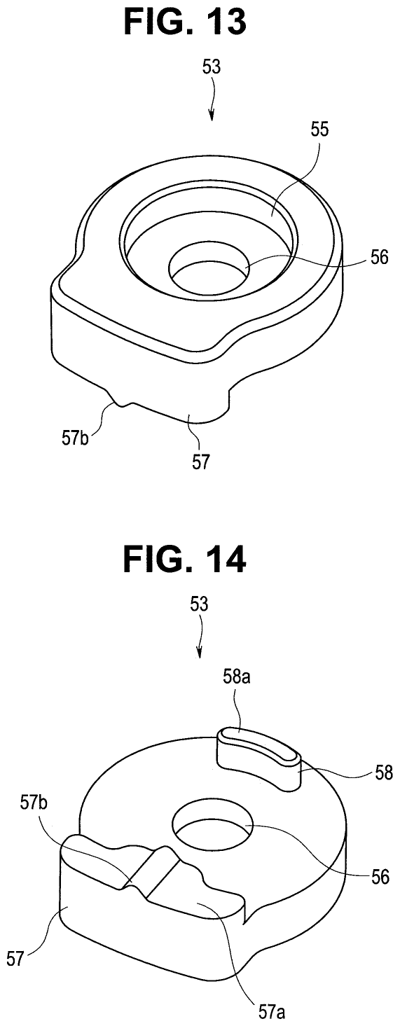

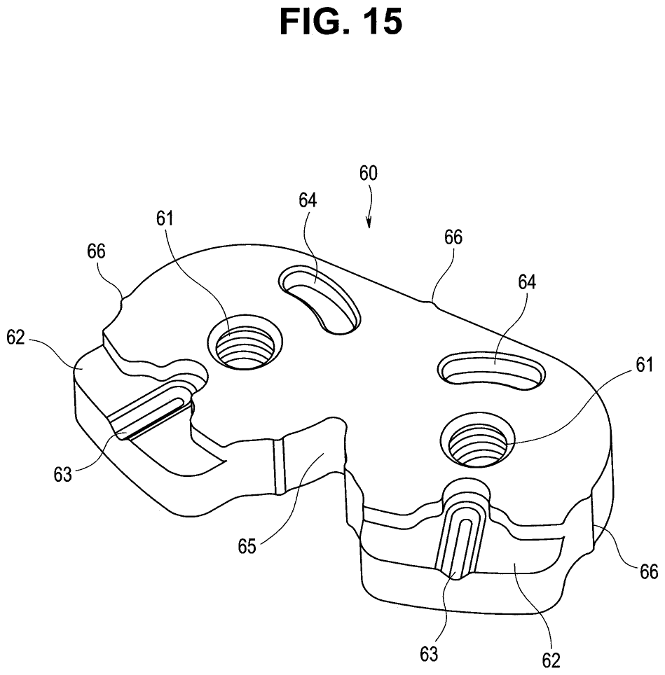

As illustrated in FIGS. 13 to 15, the wire clamp 50 includes a metal clamp 53 and a clamp base 60 as a base body. Two clamps 53 are attached to one clamp base 60.

The clamp 53 has a substantially cylindrical shape, and includes a columnar recessed portion 55 at a central portion on one surface side, a through-hole 56 formed in the center of the recessed portion 55, a wire fixing portion 57 projecting in a substantially rectangular shape from the outer peripheral portion, and a substantially arc-shaped projecting piece 58 projecting from the other surface on the opposite side relative to the wire fixing portion 57.

The other surface side of the wire fixing portion 57 is a wire pressing surface 57a serving as a wire fixing surface, and a projection 57b projected being directed toward the center of the through-hole 56 is formed at the center of the wire pressing surface 57a. The projecting piece 58 has a flat surface 58a serving as a contact surface at a projection end.

The clamp base 60 has two screw holes 61, and includes a step on one surface side where the clamp 53 is disposed facing the step; further, there are formed two wire reception surfaces 62 serving as wire fixing surfaces with which the wire fixing portions 57 of the clamps 53 are engaged. The wire reception surface 62 has a long groove 63 formed in a similar recess shape to the projection 57b of the wire fixing portion 57 while being directed toward the center of the screw hole 61.

The clamp base 60 includes a recess groove 64 formed on one surface side in a substantially arc shape, into which the projecting piece 58 of the wire fixing portion 57 is fitted, on the opposite side relative to the wire reception surface 62.

In the clamp base 60, the two wire reception surfaces 62 are formed to be separated from each other, and a V-groove 65 is formed between the two wire reception surfaces 62. The V-groove 65 is formed in order that, when the clamp base 60 is disposed while being engaged with a support 48 having a substantially triangular cross-section and erected in a clamp arrangement groove 47 of the pulleys 31 and 32 illustrated in FIG. 16, the clamp base 60 is positioned to the clamp arrangement groove 47.

The clamp base 60 is provided with a total of three projections 66, two of which are on both side portions of the outer peripheral portion and one of which is on the opposite side relative to the two wire reception surfaces 62.

These three projections 66 are formed to position the clamp base 60 so as to make the clamp base non-removable by making contact with a wall surface of the clamp arrangement groove 47 when the clamp base 60 is set in the clamp arrangement groove 47 of the pulleys 31 and 32 illustrated in FIG. 16.

In a case where the clamp arrangement groove 47 and the clamp base 60 have the same shape, the clamp base 60 cannot be attached in the clamp arrangement groove 47 due to manufacturing tolerances of the components.

Thus, the three projections 66 are formed on the clamp base 60 to form a slight gap between the wall surface of the clamp arrangement groove 47 and the side surface of the clamp base 60, thereby making it possible to attach the clamp base 60 in the clamp arrangement groove 47 within the manufacturing tolerance.

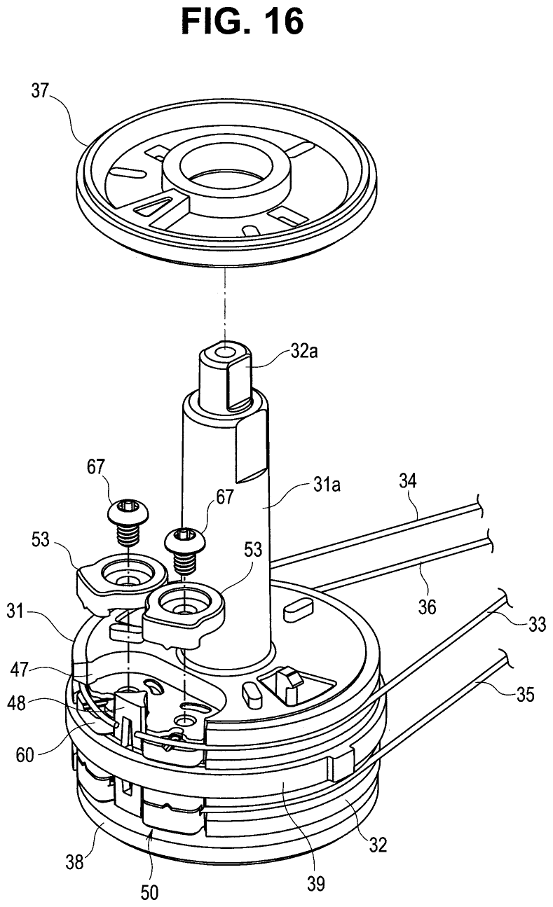

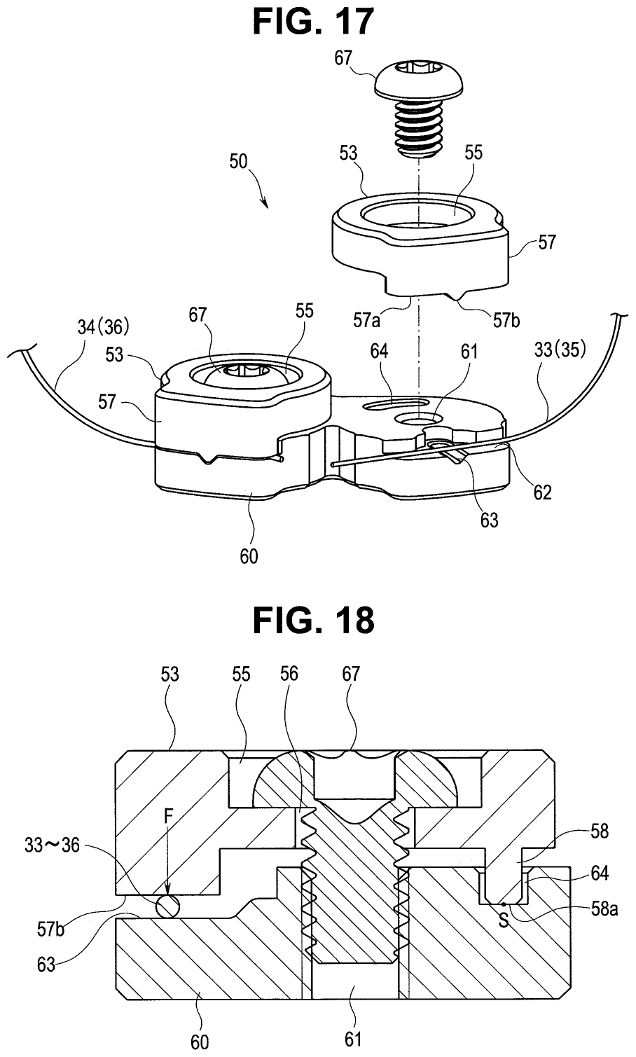

As illustrated in FIG. 17, the wire clamp 50 configured as described above fixes any of the bending operation wires 33 to 36 by clamping the clamp base 60 and the clamp 53 by tightening a screw 67.

Specifically, in a state in which the clamp base 60 is set in the clamp arrangement groove 47 of the pulleys 31 and 32, the lengths and the tensions of the bending operation wires 33 to 36 are adjusted so that the bending portion 4 of the insertion portion 21 comes to be, for example, in a linear state, and the bending operation wires 33 to 36 are set to be along on the wire reception surfaces 62 of the clamp base 60.

Subsequently, the clamp 53 is fitted to the clamp base 60 in such a manner as to press each of the bending operation wires 33 to 36 on the wire reception surface 62 by the wire fixing portion 57, and then the clamp 53 and the clamp base 60 are fastened with the screw 67.

Each of the bending operation wires 33 to 36 is in a state of being pinched between the wire pressing surface 57a of the wire fixing portion 57 and the wire reception surface 62, is pressed by the projection 57b of the wire pressing surface 57a, and is bent to be bit into the long groove 63 of the wire reception surface 62.

As illustrated in FIG. 18, in the clamp 53, the projecting piece 58 is fitted into the recess groove 64 of the clamp base 60, and by tightening the screw 67, a large pressing force F of the projection 57b of the wire pressing surface 57a directed toward the long groove 63 of the wire reception surface 62 is generated by what is called the principle of leverage, in which the flat surface 58a of the projecting piece 58 serves as a fulcrum S.

With this, the bending operation wires 33 to 36 are firmly fixed by the wire clamp 50. After the bending operation wires 33 to 36 are fixed to the wire clamp 50, excess portions of the wires are cut out.

As described above, in the present embodiment, after the lengths and the tensions of the bending operation wires 33 to 36 to be wound on the pulleys 31 and 32 are adjusted, the bending operation wires 33 to 36 are fixed by the wire clamp 50. Therefore, it is unnecessary to provide a wire adjustment mechanism of related art such as a turnbuckle for adjusting the lengths and tensions of the bending operation wires 33 to 36, and it is possible to simplify the configuration of the bending operation wires 33 to 36 extended from the pulley 31 and 32 inside the operation portion 22.

### First Reference Example

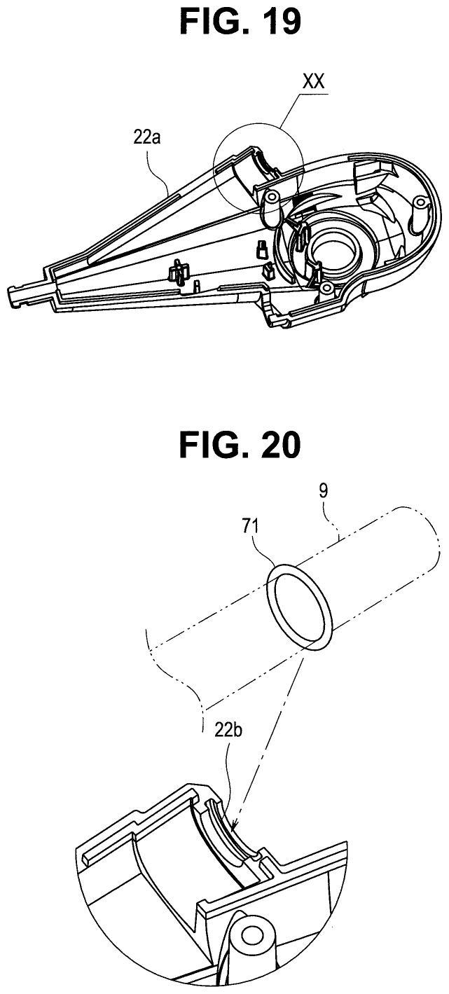

As illustrated in FIGS. 19 and 20, the baby endoscope 20 includes a groove 22b, into which a sealing member 71 such as an O-ring is fitted, at a portion formed in the outer casing member 22a of the operation portion 22 to be attached with the mouthpiece 9 of the channel insertion tube 27, so that a liquid is prevented from entering the inside of the operation portion 22 when the liquid is erroneously injected by a syringe.

### Second Reference Example

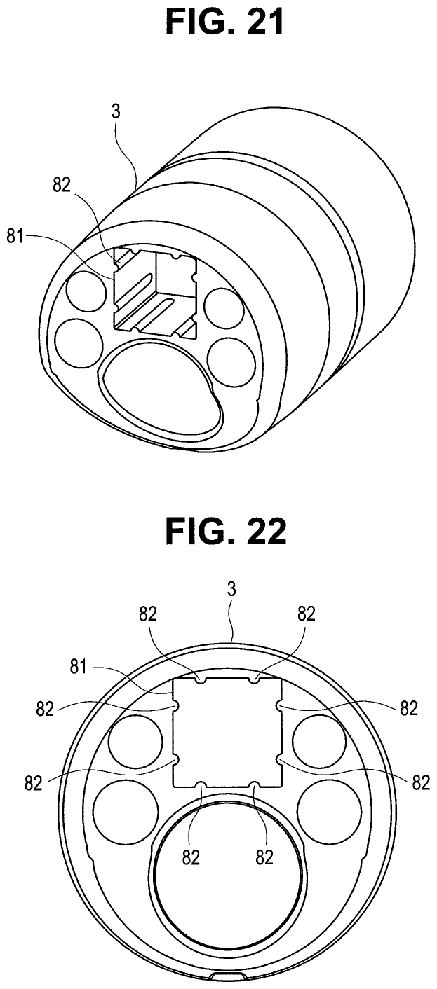

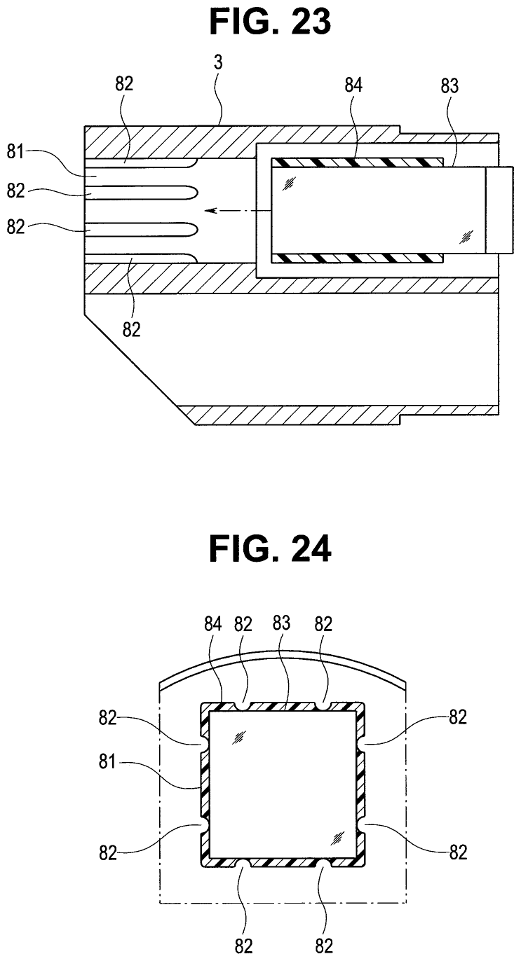

As illustrated in FIGS. 21 to 24, in the distal end portion main body 3 of the insertion portion 21, a plurality of ribs 82, in this case, a total of eight ribs 82 as projections are formed along a hole axis on wall surfaces of an opening hole 81, where an image pickup module 83 is mounted, in such a manner that two ribs are formed on each wall surface.

These eight ribs 82 are formed extending along the wall surfaces of the opening hole 81 from the distal end toward the proximal end side so as to reach an intermediate portion of the wall surfaces. That is, the eight ribs 82 are not formed in the proximal end portion of the opening hole 81.

The proximal end portion of each rib 82 has a smooth shape, in this case, has an arc-shaped cross section, so that when the image pickup module 83 is attached from the proximal end side of the opening hole 81, the image pickup module 83 is easily guided from the proximal end portion where the rib 82 is not formed, thereby improving easiness of assembling.

An adhesive agent 84 is applied to the outer surface of the image pickup module 83 from the distal end to the middle thereof, and then the image pickup module 83 is inserted into the opening hole 81. At this time, the periphery of the image pickup module 83 makes contact with the eight ribs 82, and the adhesive agent 84 flows in between the image pickup module 83 and the wall surfaces of the opening hole 81.

With this, the positioning accuracy of the image pickup module 83 with respect to the distal end portion main body 3 is improved and the easiness of assembling is improved, and the gap between the image pickup module 83 and the wall surface of the opening hole 81 is made large, whereby the adhesive agent 84 easily flows into the gap to fill the gap.

This makes it easy to control the amount of application of the adhesive agent 84 to the image pickup module 83, and makes it difficult for a space such as an air bubble to be formed in the adhesive agent 84 filling the gap between the image pickup module 83 and the wall surface of the opening hole 81. Therefore, a portion where a sterilizing gas does not reach due to a space such as an air bubble is not generated in the bonding portion of the image pickup module 83, and it is possible to prevent such a situation that the washing is difficult to be carried out.

### Third Reference Example

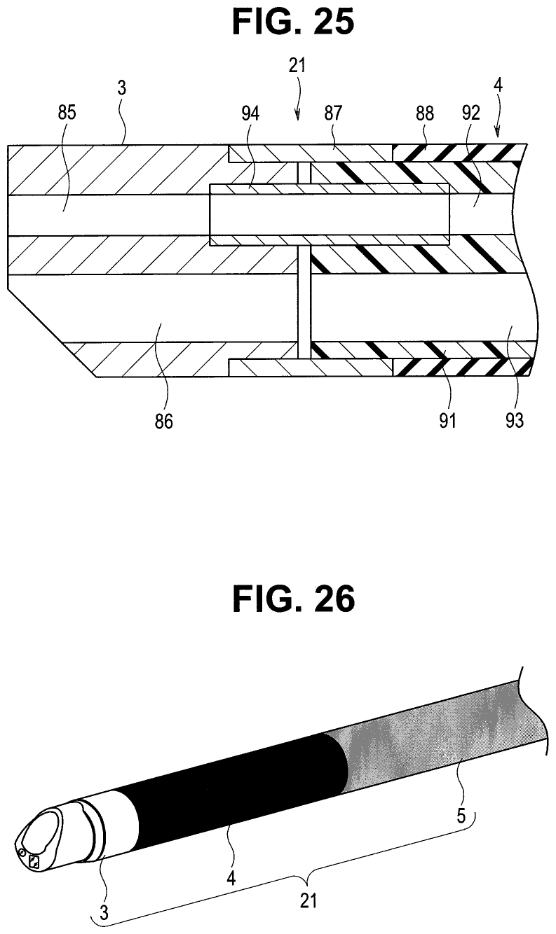

As illustrated in FIG. 25, in a case where a bending tube provided in the bending portion 4 of the insertion portion 21 is a multi-lumen tube 91, the multi-lumen tube 91 and the distal end portion main body 3 are coupled to each other by a connection tube 87. The outer periphery of the multi-lumen tube 91 is covered with an outer cover 88.

In the above configuration, a gap is generated at a connection portion where the distal end portion main body 3 and the multi-lumen tube 91 face each other, and a liquid flows from a liquid supply lumen 92 of the multi-lumen tube 91, through which a liquid such as a contrast medium agent or a physiological saline solution flows, into a lumen 93 such as another treatment instrument channel formed in the multi-lumen tube 91, a channel hole portion 86 of the distal end portion main body 3, or the like.

Thus, by adopting a configuration in which a liquid supply channel 85 of the distal end portion main body 3 and the liquid supply lumen of the multi-lumen tube 91 are connected to communicate with each other by a tube member 94 made of metal or resin, the liquid flowing through the liquid supply lumen 92 is prevented from flowing into other channels, lumens, and the like.

### Fourth Reference Example

As illustrated in FIG. 26, by making the external appearance colors of the bending portion 4 and the flexible tube portion 5 of the insertion portion 21 of the baby endoscope 20 different from each other, the bending state of the bending portion 4 of the baby endoscope 20 is easily recognized by a scope image of the mother endoscope 10, and the insertion operation into the subject may be easily performed.

When the insertion portion 21 of the baby endoscope 20 has a light color such as white, halation or the like occurs due to reflection of illumination light of the mother endoscope 10. Even in a case where halation does not occur, when the insertion portion 21 of the baby endoscope 20 has a light color, the light control function of the mother endoscope 10 works and the amount of illumination is suppressed in the case of the insertion portion 21 being located in an image pickup area. As a result, with the mother endoscope 10, a sufficient amount of illumination light cannot reach the major duodenal papilla.

Because of this, in order not to inhibit the visual recognition of the major duodenal papilla in the scope image of the mother endoscope 10, the insertion portion 21 of the baby endoscope 20 has a dark color based on black as an external appearance color of the bending portion 4, and the flexible tube portion 5 has a dark gray color.

The invention described in the above embodiments and modifications is not limited to the embodiments and modifications, and various modifications can be made without departing from the scope of the invention in the implementation stage. Furthermore, the above-described embodiments and modifications include inventions at various stages, and various inventions can be extracted by appropriately combining a plurality of constituent elements disclosed.

For example, even when some constituent elements are deleted from all the constituent elements shown in the embodiments and the modifications, in a case where the described problems can be solved and the described effects can be obtained, the configuration from which the constituent elements are deleted can be extracted as an invention.

What is claimed is:

1. An endoscope, comprising:
an endoscope insertion portion provided with a bending portion;
an operation portion connected with the endoscope insertion portion;
an operation member provided in the operation portion and configured to perform bending operation on the bending portion;
a pulley provided in the operation portion, having an outer diameter larger than an inner diameter of the endoscope insertion portion, and configured to wind a wire connected to the bending portion; and
a wire guard that is arranged to cover a predetermined range of an outer peripheral portion of the pulley, and includes a guide wall arranged to extend within a range of a radius of the pulley from a contact point position at which the wire in contact with the pulley is separated from the pulley in a longitudinal axis direction of the operation portion,
wherein the wire guard includes,
two members each formed in a substantially semicircular plate shape, and
coupling parts formed in arc shapes extending from both end portions of the two members and configured to couple the two members.

2. The endoscope according to claim 1,
wherein an extended end of the guide wall is disposed on a side of the endoscope insertion portion relative to the contact point position.

3. The endoscope according to claim 2,
wherein the guide wall is disposed on the side of the endoscope insertion portion relative to a rotation center of the pulley.

4. The endoscope according to claim 2,
wherein the guide wall is disposed in parallel along an axis of the wire.

5. The endoscope according to claim 2,
wherein the wire guard is disposed including a gap, between an inner surface of the wire guard and the outer peripheral portion of the pulley, equal to or smaller than an outer diameter of the wire.

6. The endoscope according to claim 1,
wherein a coupling section of the coupling parts is disposed on a side of the endoscope insertion portion relative to the pulley.

7. The endoscope according to claim 2, further comprising:
a substrate that is disposed in the operation portion and on which an electronic component is mounted; and
a plate provided between the wire and the substrate.

8. The endoscope according to claim 2, further comprising:
a pulley cover overlapping the pulley,
wherein the pulley cover includes a locking member configured to engage with a locking claw protruding from one surface of the pulley, and is attached and fixed to the pulley.

9. The endoscope according to claim 1,
wherein the wire guard is disposed including a gap, between an inner surface of the wire guard and the outer peripheral portion of the pulley, equal to or smaller than an outer diameter of the wire.
